(19) United States
(12) Patent Application Publication
(10) Pub. No.: US 2020/0152319 A1
(43) Pub. Date: May 14, 2020

(54) SCHEDULING A TASK FOR A MEDICAL PROFESSIONAL

(71) Applicant: KONINKLIJKE PHILIPS N.V., EINDHOVEN (NL)

(72) Inventors: Gijs Geleijnse, Geldrop (NL); Ana Jorge Rodrigues de Moura Leitao, Eindhoven (NL); Marco van Leeuwen, Eindhoven (NL); Jennifer Caffarel, Eindhoven (NL)

(21) Appl. No.: 16/617,032

(22) PCT Filed: May 17, 2018
(86) PCT No.: PCT/EP2018/062846

§ 371 Date: Nov. 26, 2019

Related U.S. Application Data
(60) Provisional application No. 62/511,394, filed on May 26, 2017.

(51) Int. Cl. G16H 40/20; G16H 10/60; G16H 70/20; G16H 50/70; G16H 20/00; G06N 20/00; G06N 7/00; G06N 5/04

(52) U.S. Cl. CPC G16H 40/20; G16H 10/60; G16H 70/20; G06N 5/048; G16H 20/00; G06N 20/00; G06N 7/005; G16H 50/70

(57) ABSTRACT

According to an aspect, there is provided a computer-implemented method of scheduling a task for a medical professional. The method has steps of obtaining patient characteristics associated with a patient and obtaining test subject characteristics associated with a plurality of test subjects. A task to be performed in relation to a patient is then identified based on the patient characteristics, the test subject characteristics and one or more tasks performed on the test subjects. The method then comprises scheduling, using a processor, the identified task for the medical professional. A computer program product is also disclosed.

### TECHNICAL FIELD

Various embodiments described herein relate to methods and apparatus for scheduling tasks for a medical professional.

### BACKGROUND

Clinical pathways are designed with the aim of offering standardized, value-based healthcare to a specific group of patients. Such pathways provide step-by-step guidance for a multi-disciplinary team to offer care for a specific disease (e.g. colon cancer) or situation (e.g. a patient entering an Emergency Department with chest pain).

In a typical care setting deploying pathways, paper copies of the clinical pathways are created with flow charts that are used to describe the sequence of tasks in the clinical pathway and the points in the pathway where a medical professional needs to make a decision. Such paper-based systems make scheduling and co-ordination of tasks between different departments more difficult, particularly if paper records are not cross-checked with any electronic information held on the patient for the most up to date information. Also, paper systems do not take the care teams' workloads relating to other patients into account.

Where the pathways represent a patient and condition-oriented view on care, they are part of a complex combination of tasks and processes. A medical professional will have various tasks outside the care of a single patient in a single pathway. Patients may also have needs and conditions outside the described pathway. Co-ordinating and scheduling tasks for medical professionals from these different sources is therefore often challenging.

### SUMMARY

As noted above, clinical pathways provide high-level guidance on the steps that need to be followed by medical professionals in certain situations or when treating particular medical conditions. These have the advantage of standardising care, but have a number of drawbacks. Firstly, clinical pathways are described at a fairly high-level such that each task, (or action block) in the clinical pathway may involve more than one medical professional. In such a case, each task in the clinical pathway may represent multiple tasks involving different medical professionals in a medical facility. To take a simple example, a task of “administer drug X” as described in a clinical pathway may require the steps of i) a pharmacist preparing drug X, ii) a ward clerk collecting drug X from the pharmacist, and iii) a nurse administering drug X to the patient. As such, tasks in a clinical pathway are not always split into components small enough so that they can be assigned to an individual medical professional.

Secondly, a clinical pathway may ‘branch’ and have two or more routes that may be assigned to the patient according to the needs/circumstances of the patient. In this case, a medical professional must assign the patient to a particular branch before the tasks in the branch can be scheduled. This lack of certainty as to the direction of patient care and required tasks makes resource planning more difficult.

Thirdly, the implementation of tasks in a clinical pathway may vary between medical facilities due, for example, to different medical facilities having different internal policies or workflows. Returning to the simple example above, in a first hospital, it may be the pharmacist who prepares drug X whereas, in a second hospital, a ward clerk may need to contact an outside supplier to provide drug X to the patient. It is therefore not feasible to have detailed clinical pathways that are suitable for all hospitals.

Fourthly, medical facilities or even medical professionals within a particular hospital may choose to implement clinical pathways slightly differently, depending on individual practice. They may, for example, routinely supplement tasks specified in a clinical pathway with additional tasks or checks, for example, to further improve the clinical pathway, or according to specialist knowledge in a field. As described above, these sorts of practices may be highly individualised and vary between districts, medical facilities and/or individual medical professionals.

With such variation and so many unknowns, it is difficult to create accurate schedules for medical staff and to regulate workloads. There is therefore a need to improve the management of clinical pathways and the allocation of tasks to medical professionals in the medical facility environment.

Therefore, according to a first aspect, there is a computer-implemented method of scheduling a task for a medical professional. The method comprises obtaining patient characteristics associated with a patient; obtaining test subject characteristics associated with a plurality of test subjects; identifying a task to be performed in relation to the patient based on the patient characteristics, the test subject characteristics and one or more tasks performed on the test subjects; and scheduling, using a processor, the identified task for the medical professional.

In some embodiments, a clinical pathway is assigned to the patient, the clinical pathway having a plurality of branches, each branch including a plurality of tasks. In these embodiments, the step of identifying comprises: predicting that a first branch in the clinical pathway will be followed in relation to the patient, based on the patient characteristics, the test subject characteristics and the branches followed for each of the test subjects.

In some embodiments, predicting that a first branch in a clinical pathway will be followed comprises assigning a probability that a first branch will be followed in relation to the patient, based on i) a proportion of the test subjects who followed the first branch of the clinical pathway; ii) the patient characteristics; and iii) the test subject characteristics.

In some embodiments, assigning a probability further comprises matching the patient to a subset of the test subjects with similar test subject characteristics to the patient characteristics. In some embodiments, similar comprises: the test subject characteristics for the subset being identical to the patient characteristics; the test subject characteristics for the subset being within a predefined threshold of the patient characteristics; the test subject characteristics for the subset being considered a fuzzy match; or the test subject characteristics being considered similar to the patient characteristics by a machine learning algorithm.

In some embodiments, the step of scheduling comprises, scheduling a task from the first branch in the clinical pathway if the probability is higher than a threshold.

In some embodiments, the step of scheduling comprises notifying the medical professional that the identified task is a predicted task.

In some embodiments, the patient characteristics comprise details of a clinical pathway assigned to the patient; the test subject characteristics comprise details of one or more clinical pathways assigned to each of the test subjects; and the step of identifying a task comprises: identifying a task to be performed in relation to the patient based on tasks performed for a subset of the test subjects who have been assigned to the same clinical pathway as that assigned to the patient.

In some embodiments, each clinical pathway includes a plurality of tasks to be performed; and the identified task is a task determined to have been performed in relation to the subset of test subjects, in addition to, or instead of, one or more tasks included in the clinical pathway assigned to the patient.

In some embodiments, the identified task is not a task included in the clinical pathway assigned to the patient.

In some embodiments, the method further comprises: obtaining a list of tasks included in the clinical pathway assigned to the patient; comparing the identified task to the list of tasks; and determining not to schedule the identified task if the identified task is one of the tasks in the list of tasks.

In some embodiments, the step of identifying further comprises using a machine learning algorithm to identify the task, based on the patient characteristics, the test subject characteristics and tasks performed on the one or more test subjects.

In some embodiments, the method further comprises: receiving additional patient characteristics associated with the patient; and determining whether the identified task should be performed based on the additional patient characteristics, the test subject characteristics and one or more tasks performed on the test subjects.

In some embodiments, the method further comprises: removing the identified task from the schedule of the medical professional, if it is determined that the identified task should not be performed.

In some embodiments, the step of identifying a task comprises comparing the patient characteristics to the test subject characteristics.

According to a second aspect, there is a computer program product comprising a non-transitory computer readable medium, the computer readable medium having computer readable code embodied therein, the computer readable code being configured such that, on execution by a suitable computer or processor, the computer or processor is caused to perform the method of any of the preceding embodiments.

### BRIEF DESCRIPTION OF THE DRAWINGS

For a better understanding, and to show more clearly how it may be carried into effect, reference will now be made, by way of example only, to the accompanying drawings, in which:

FIG. 1 is a simplified schematic of an apparatus according to an embodiment;

FIG. 2 is a flowchart showing an example method according to an embodiment;

FIG. 3 is a simplified schematic of an example of a clinical pathway according to an embodiment; and

FIG. 4 is a flowchart showing an example method according to an embodiment.

### DETAILED DESCRIPTION

The description and drawings presented herein illustrate various principles. It will be appreciated that those skilled in the art will be able to devise various arrangements that, although not explicitly described or shown herein, embody these principles and are included within the scope of this disclosure. As used herein, the term “or” refers to a non-exclusive or (i.e., and/or), unless otherwise indicated (e.g., “or else” or “or in the alternative”). Additionally, the various embodiments described herein are not necessarily mutually exclusive and may be combined to produce additional embodiments that incorporate the principles described herein.

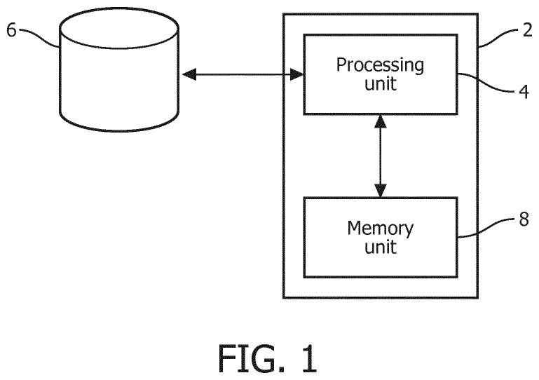

FIG. 1 shows a simplified schematic of an apparatus 2 for scheduling a task for a medical professional, according to embodiments of the present disclosure. The apparatus 2 includes a processing unit 4 that is in communication with a database 6 which holds a dataset including test characteristics associated with a plurality of test subjects. The processing unit 4 can, as will be described in more detail below, obtain patient characteristics associated with a patient, obtain test subject characteristics associated with a plurality of test subjects and identify a task to be performed in relation to the patient based on the patient characteristics, the test subject characteristics and one or more tasks performed on the test subjects. The processor can then schedule the identified task for the medical professional.

In some embodiments, the apparatus 2 is a computing device, such as a laptop computer, a desktop computer, a smartphone, a tablet computer or some other portable electronic device. The database 6 may be contained within the apparatus 2 or may be remote from the apparatus 2. For example, the database 6 may be stored on a remote server. Queries run by the processing unit 4 on the database 6 may therefore be executed locally in the apparatus 2, or remotely.

The processing unit 4 can be implemented in numerous ways, with software and/or hardware, to perform the various functions described below. The processing unit 4 may comprise one or more microprocessors or digital signal processor (DSPs) that may be programmed using software or computer program code to perform the required functions and/or to control components of the processing unit 4 to effect the required functions. The processing unit 4 may be implemented as a combination of dedicated hardware to perform some functions (e.g. amplifiers, pre-amplifiers, analog-to-digital convertors (ADCs) and/or digital-to-analog convertors (DACs)) and a processor (e.g., one or more programmed microprocessors, controllers, DSPs and associated circuitry) to perform other functions. Examples of components that may be employed in various embodiments of the present disclosure include, but are not limited to, conventional microprocessors, DSPs, application specific integrated circuits (ASICs), and field-programmable gate arrays (FPGAs).

In various implementations, the processing unit 4 may be associated with or comprise one or more memory units 8 such as volatile and non-volatile computer memory such as RAM, PROM, EPROM, and EEPROM. The processing unit 4 or associated memory unit 8 can also be used for storing program code that can be executed by a processor in the processing unit 4 to perform the method described herein. The memory unit 8 can also be used to store data retrieved from the database 6.

It will be understood that FIG. 1 constitutes, in some respects, an abstraction and that the actual organization of the components of the apparatus 2 may be more complex than illustrated. Furthermore, the apparatus 2 may comprise additional components not specifically illustrated in FIG. 1, for example, the apparatus 2 may comprise one or more devices for enabling communication with a user such as a medical professional. For example, the apparatus 2 may include a display, a mouse, and/or a keyboard for receiving user commands.

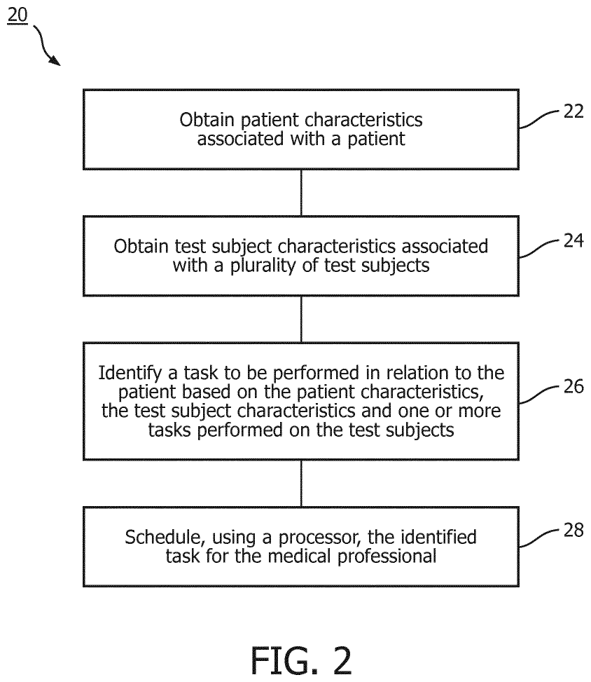

Turning now to FIG. 2, FIG. 2 shows a flow chart of a method 20 for scheduling a task for a medical professional. The method 20 may be performed using an apparatus such as the apparatus 2. The method 20 comprises the steps of: obtaining 22 patient characteristics associated with a patient; obtaining 24 test subject characteristics associated with a plurality of test subjects; identifying 26 a task to be performed in relation to the patient based on the patient characteristics, the test subject characteristics and one or more tasks performed on the test subjects; and scheduling 28, using a processor, the identified task for the medical professional.

In some embodiments, the step of obtaining 22 patient characteristics comprises obtaining patient characteristics from a database, such as the database 6 in FIG. 1. The processing unit 4 may query the database 6 for information about the patient. The database may be stored locally on the device 2, or alternatively it may be stored remotely.

In a similar manner, the step of obtaining 24 test subject characteristics associated with a plurality of test subjects may comprise obtaining the test subject characteristics from a database, such as the database 6 in FIG. 1. The processing unit 4 may thus query the database 6 for information about the test subjects. It should be noted that the patient characteristics and the test subject characteristics may be stored in the same, or different locations. For example, there may be more than one database 6.

Patient characteristics include any information about a patient, in relation to whom it is envisaged that tasks will need to be scheduled (i.e. allocated to one or more medical professionals). Examples of patient characteristics include, but are not limited to, information about health conditions, “vital signs” measurements, details of previous treatments or previous medical conditions, demographic data about the patient and/or details of the patient's lifestyle. As an example, the patient characteristics might include the names of one or more medical professionals assigned to care for the patient, the patient's ethnicity, the patient's age, the reason for admittance to the medical facility and the patient's blood pressure and heartrate measurements.

The plurality of test subjects might include other patients that have been treated or are currently being treated. The test subjects may be associated with (e.g. treated by) the same medical professional as the patient. In some embodiments, the test subjects are treated by different medical professionals to the patient. The test subjects may be associated with (e.g. treated in) the same and/or different medical facilities and the same and/or different groups of medical facilities (i.e. medical facilities in different parts of the country, or medical facilities managed by different authorities or companies). In other words, the test subjects may provide a set of previous examples of how other patients have been treated, for example, by a particular medical professional, a particular medical facility or a particular group of medical facilities.

The test subject characteristics include any information about the test subjects, including but not limited to, information about health conditions, “vital signs” measurements, details of previous treatments or previous medical conditions, demographic data about the test subjects and/or details of the test subjects' lifestyles. The test subject characteristics may be the same as, or different to, the patient characteristics.

Once the patient characteristics and the test subject characteristics are obtained (e.g. at step 24), the method then comprises identifying 26 a task to be performed in relation to the patient based on the patient characteristics, the test subject characteristics and one or more tasks performed on the test subjects.

A task is any action that can be performed in relation to the patient, including but not limited to, actions relating to the care of the patient (e.g. “take the patient's blood” or “take the patient's vital signs measurements”) or to the administrative tasks associated with the patient's care in the medical facility (e.g. “create a new electronic medical record for the patient” or “create an invoice”). Preferably, a task is actionable (e.g. can be performed) by an individual medical professional. However, it will be understood that, in some embodiments, a task may require the presence of one or more medical professionals (e.g. a task of “turn the patient” may require two or three medical professionals working together).

In some embodiments, the step of identifying a task comprises comparing the patient characteristics to the test subject characteristics, in order to identify test subjects (e.g. previous patients) with the same or similar circumstances and/or conditions. Once a subset of test subjects who have the same or similar circumstances and/or conditions is found, the tasks in the clinical pathways used for those patients might be used to predict (or guide) the treatment plan for a patient currently being treated. In other words, information about the current patient and information about previous patients (test subjects) is compared in order to identify or predict tasks that are also likely to be required in respect of the patient currently being treated.

In some embodiments, the step of identifying comprises identifying an applicable pathway for the patient. The method may then comprise, for each pathway, identifying the current position along the pathway (i.e. the point in the pathway that has been reached by the medical professionals assigned to the patient).

Details of the current position may be stored, for example, electronically, in an electronic record held for the patient. Alternatively, the current position may be derived from information about the tasks that have already been performed on the patient. Information about the tasks that have already been performed may be derived, for example, from any notes made about the patient's care (including hand-written notes) and/or the electronic medical record (EMR) for the patient.

These identified tasks can then be scheduled for the medical professional to perform. In some embodiments, scheduling comprises allocating a task to a medical professional. The medical professional may have a list of tasks that they are to perform. In some embodiments, scheduling may comprise adding the identified task to the list of tasks that are to be performed by the medical professional. In some embodiments, the step of scheduling can comprise allocating a time or time period in which the task should be performed. In some embodiments, the time period is added to a diary or calendar that contains details of all of the tasks that are assigned to the medical professional. In some embodiments, the task is associated with a workload (e.g. an expected time required to complete the task), for example in minutes or seconds.

In an example where medication is administered to a patient, the medication should be ordered prior to administration. Tasks of ordering a medication and administering the medication may therefore be scheduled sequentially for the relevant medical professions. In some examples, strict time windows may be planned (e.g. between 13:00 and 13:10, with a workload of 4 minutes), whilst in other examples, tasks may be allocated to take place within larger time windows, for example, tasks may be allocated to a particular morning, or to sometime (at the discretion of the medical professional) between 03:00 and 13:00, with a workload of 3 minutes.

This method of scheduling tasks has significant advantages, because, by analysing tasks performed on previous patients, information can be gained about the likely pattern of tasks that will be performed on current and future patients. As described above, clinical pathways which specify a list of tasks that should be performed for a patient are used in medical facilities to help ensure best practice is followed. However, these are often only described at a high level without details of how the high-level tasks may be divided and implemented in a particular medical facility. Using the method described above, greater visibility can be obtained regarding how clinical pathways are implemented within individual medical facilities. For example, if a medical facility regularly supplements tasks of a clinical pathway with additional tasks, then these can be foreseen and planned for ahead of time. In this way, tasks can be predicted and scheduled, providing increased visibility on upcoming workloads for medical professionals.

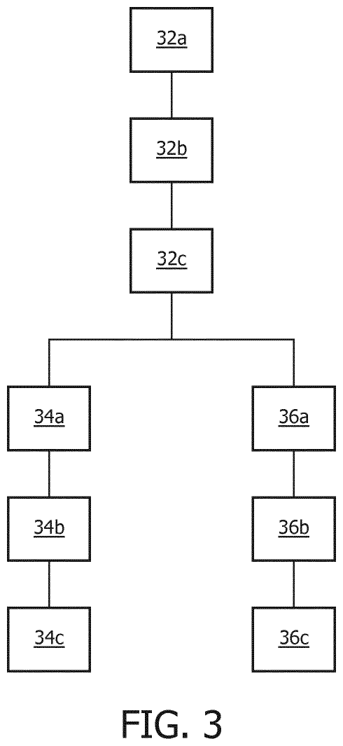

In some embodiments, a clinical pathway can split into two or more branches of tasks. For example, as shown in FIG. 3 a clinical pathway may specify a first set of tasks, 32a, 32b and 32c to be performed, after which, the clinical pathway may branch, and tasks 34a, 34b and 34c will be assigned as tasks that are to be performed in relation to the patient (e.g. if a first branch is followed) or tasks 36a, 36b and 36c will be assigned (e.g. if a second branch is followed). It will be understood that the number of tasks (e.g. action blocks) in FIG. 3 is merely exemplary and that a clinical pathway may include different numbers of tasks to those shown in FIG. 3 and/or different numbers of branches.

In some embodiments, therefore, a clinical pathway is assigned to the patient, the clinical pathway having a plurality of branches where each branch comprises a plurality of tasks. The method may comprise predicting that a first branch in the clinical pathway will be followed in relation to the patient, based on the patient characteristics, the test subject characteristics and the branches followed for each of the test subjects.

In other words, the branch assigned to the patient can be predicted from the branches assigned to previous patients (e.g. test subjects), taking into account characteristics of the patient and the previous patients (e.g. test subjects).

In some embodiments, the step of predicting that a first branch will be followed in relation to the patient comprises comparing the patient characteristics to the test subject characteristics to obtain a subset of the test subjects with similar or matching characteristics to the patient and predicting that a first branch will be followed in relation to the patient, based on the branches followed by the similar test subjects.

A test subject might be considered to be “similar” to the patient (i.e. the patient currently being treated) if they have one or more matching characteristics (e.g. they may have the same condition; for example, the patient and the similar test subject may both have diabetes). In some embodiments, the test subject may have similar characteristics if values of the test subject characteristics are within a threshold tolerance of values of the patient characteristics. For example, the test subject may be considered to be similar to the patient (i.e. the patient currently being treated) if particular values of the test subject's characteristics are within a predefined percentage of the values of corresponding patient characteristics. In some embodiments, the predefined percentage may be 10 percent.

In some examples, a test subject might be considered similar if they are deemed similar by a fuzzy matching algorithm or a machine learning algorithm. In this sense, a machine learning algorithm may produce a model that calculates, for a given set of characteristics, the most closely matching test subjects.

Once a subset of test subjects has been obtained, a probability can be assigned that a first branch will be followed in relation to the patient, based on the proportion of similar test subjects who were assigned to the first branch. For example, if 90 percent of similar patients were assigned to a first branch in a clinical pathway then it may be said that there is a 90 percent likelihood that a subsequent patient with similar characteristics will also be assigned to the first branch.

Once a patient is assigned to a branch by a medical professional (i.e. once the branch becomes known), the likelihood that the tasks in that branch will be performed approaches 1 (i.e. it is certain, barring unforeseen circumstances such as the patient being discharged, or moved, that the tasks in that branch will be performed).

Tasks can then be scheduled, based on the assigned probabilities of each branch. In some embodiments, a task is scheduled if the probability is above a threshold. For example if it is more than 80 percent likely that the patient will be assigned to a branch, then the tasks in that branch may be assigned to the relevant medical professionals. It will be appreciated that the threshold may be set at any level, according to the requirements of the medical facility and that, in some embodiments, the predicted tasks of all branches may be scheduled, regardless of the relative probabilities of the branches.

In some embodiments, tasks will be scheduled with different certainties according to the probability that they will be performed. For example, tasks with a probability of being performed of 1 will be scheduled for the medical professional, whilst tasks with lower probabilities (e.g. predicted tasks) may be provisionally scheduled. Provisionally scheduled tasks, may, for example, be accompanied by a notification, or alert to the medical professional to indicate that the scheduled task is not certain. In some embodiments, a visual indication may be given to indicate the certainty of the task (e.g. tasks with a probability of needing to be completed of >95 percent may be indicated in a first colour, whilst tasks with a probability of 80-95 percent may be in a second, different, colour).

In this way, tasks that form part of a branch of a clinical pathway can be predicted and scheduled before a decision is made by the medical professional as to whether that particular branch of the clinical pathway is to be followed. This provides more accurate foresight into upcoming workloads of medical professionals than would otherwise be possible.

Turning now to another embodiment, as described above, clinical pathways often provide high-level guidance of what should be done for a patient. However, this high-level guidance often is not divided into actionable steps to be performed by individual medical professionals or groups of medical professionals involved in a patient's care. Furthermore, it may not be possible to divide the clinical pathway into actionable steps, if, for example, different medical facilities or different medical professionals within the same medical facility have different preferred ways of complying with the clinical pathway. Lastly, there may be other tasks related to, or completely separate from, any clinical pathways assigned to the patient, that nonetheless are performed on patients in various situations in a particular medical facility.

Therefore, in some embodiments, the method 20 is used to identify such tasks in relation to a particular patient. In one embodiment of the method 20, the patient characteristics and the test subject characteristics may comprise details of clinical pathways assigned to the patient and each of the test subjects respectively. In this embodiment, the step of identifying a task comprises identifying 26 a task to be performed in relation to the patient based on tasks performed for a subset of the test subjects who have been assigned to the same clinical pathway as that assigned to the patient.

For example, if all test subjects who were assigned to a sepsis clinical pathway also happened to have their blood tested every 24 hours, then, for a new patient following the sepsis pathway, a task of “take the patient's blood” may be identified and scheduled (e.g. assigned) to a relevant medical professional to be performed every 24 hours, irrespective of whether this task is a task explicitly stated in the sepsis pathway.

In some embodiments, tasks identified in this way (e.g. by comparison with tasks that have been performed on other similar patients) may be flagged as predicted tasks. In some examples, a medical professional may need to authorise such identified tasks before they are scheduled.

In this way, the identified task may be a task that is performed instead of, or in addition to one or more tasks specifically stated in the clinical pathway. The task may thus be a task that is individualised to the standard practice of the medical facility associated with the patient.

Thus, the identified task may be a task that is not explicitly specified in the clinical pathway. The task may be a task that is separate from the clinical pathway but commonly performed in a particular medical facility, for example, due to the specific knowledge and experience of medical practitioners in that field. For example, particular practitioners may routinely prefer to check for diabetes in patients with high BMI (Body Mass Index)—even if this isn't part of a clinical pathway, in which case, “check diabetes” may be added to the task list of the relevant medical practitioner for all patients that are assigned to an obesity-related clinical pathway.

In some embodiments, the step of identifying a task further comprises using a machine learning algorithm to identify the task. Examples of suitable machine learning algorithms include Decision Trees, Support Vector Machines, Neural Networks and Deep Learning algorithms. In such embodiments, the test subject characteristics and tasks performed on the test subjects are provided as input parameters to the machine learning algorithm. The machine learning algorithm produces a model that can be used to predict, from a set of characteristics (e.g. the patient characteristics), the tasks that will be performed for the patient. The model may be improved over time by expanding the number of test subjects and/or providing a larger number of characteristics (i.e. increasing the number of input parameters to the machine learning algorithm).

In some embodiments, as care progresses, new characteristics (e.g. information) about the patient is obtained and this can be used to improve the forecasted tasks that need to be performed in relation to the patient. The method can therefore further comprise receiving additional patient characteristics associated with the patient, and determining whether the identified task should be performed based on the additional patient characteristics, the test subject characteristics and one or more tasks performed on the test subjects.

If it is determined that the task should no longer be performed, then the method may comprise removing the identified task from the schedule of the medical professional.

In some embodiments, the additional patient characteristics may be new information about the patient and/or the patient's care. For example, the additional characteristics may be one or more of: a new clinical pathway assigned to the patient, information about a decision to assign a particular branch of a clinical pathway to the patient, a new vital signs measurement for the patient and/or the results of a test performed on the patient.

In some embodiments, predicting and assigning tasks using any of the methods above may lead to duplication of tasks. Therefore, in some embodiments, the method may further comprise identifying a duplicated task in a schedule. The method may further comprise removing a duplicated task from a schedule of a medical professional.

Failure to adhere to clinical pathways is a major problem in critical care. Even when staff are fully aware of the details of the pathway, the patients on a pathway and the actions associated with the pathway, they may fail to adhere due to time pressure, workload, bad planning or forgetfulness. The method herein also has the advantage of fully embedding the tasks of the medical professional into a schedule. The pathway is not presented in isolation, or as a series of abstract tasks that need to be done by the medical professionals in the medical facility as a whole, but as an integrated part of the work of the individual medical professional. This method also allows predictions of work overload such that appropriate resources can be allocated.

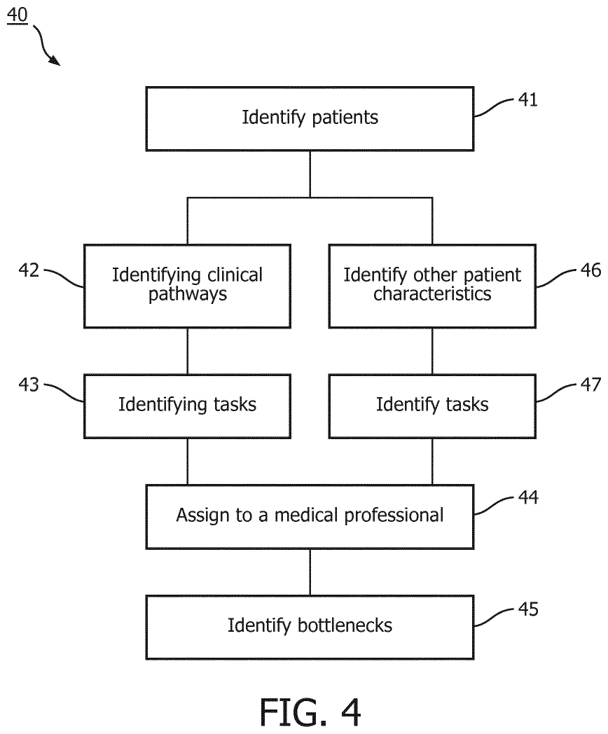

Turning now to FIG. 4, a flowchart sets out steps of a method 40 that uses and combines various elements of the embodiments described above and describes how the methods may be applied to scheduling tasks for a plurality of patients in a medical facility.

In a first step, the method 40 comprises identifying 41 patients who will utilise a particular medical facility in a defined period of time. In some embodiments, a list of patients is generated. The list of patients is based on the patients that are foreseen to utilize the services of the healthcare provider in a defined period. For example, the list may comprise all patients currently admitted, in addition to all patients scheduled for admission the following day. This is merely exemplary, however, and the list could also include patients scheduled to be admitted within any upcoming time frame, for example, the defined period may be the next two days, the next week, the next fortnight etc.

In some embodiments, a second list can also be created, containing information on patients who may be transferred to or from the medical facility in question and who may be admitted within the defined time period. This can help in the planning of transfers between departments (e.g. a neonatal ICU may want to keep track of all pregnant patients admitted to the medical facility as a prevention planning measure, to ensure they are ready with correct resources if, for example, a patient were to give birth prematurely due to some complication resulting from a non-pregnancy related treatment).

When a list of patients is identified, tasks for each of the identified patients can be identified in different ways, as exemplified in the two paths (a first path including steps 42 and 43 and a second path including steps 46 and 47) in FIG. 4.

In the first (left-hand) path, tasks may be derived from the clinical pathways assigned to the patient. A step of identifying 42 clinical pathways assigned to each of the patients may comprise identifying clinical pathways from the electronic medical record (EMR) of the patient, or from other notes or records kept on the patient.

Once clinical pathways have been identified, tasks are identified 43 for each pathway that is assigned to each patient. In some embodiments, tasks may be identified using the method 20 as shown in FIG. 2 and described in the examples above. In some embodiments the clinical pathways identified in step 42 may be one or more of the patient characteristics associated with the patient, as described in step 22 of the method 20.

In some embodiments, one or more of the tasks identified in step 43 may be predicted tasks. As described in detail in the examples above, a clinical pathway may branch into two or more series of tasks that may be performed on a patient, depending on the branch that is assigned to the patient. Before a branch of a clinical pathway is assigned to a patient, tasks from one or more branches may be predicted and provisionally scheduled for a medical professional, using the method described above with respect to the method 20.

In some embodiments, tasks may be derived from the clinical pathways identified in step 42 in other ways, in addition or instead of, those described with respect to the method 20 above. For example, as described above, a task in a clinical pathway may be described at a high level. Such high-level tasks may be broken down using a look-up table that can be used to divide the high-level tasks into tasks that can be allocated to an individual. For example, if a pathway says to “administer X ml of medicine Y”, this step may be divided into the actions of prescribing and ordering the medication, making sure the medication is available at the point of care and administrating the medication at a required time. Using this look-up table, the tasks related to the high-level task may be determined. In some embodiments therefore, the step of identifying 42 tasks may comprise identifying a high-level task in a look-up table and identifying tasks associated with the high-level task from tasks listed in the look-up table.

In addition to tasks derived from the clinical pathways, patients have treatment needs that are not described in clinical pathways. Therefore, in addition to tasks that are identified from clinical pathway data, tasks may also be identified from other characteristics, such as the results of diagnostic procedures performed on the patient. Therefore, the method 40 may comprise a step of identifying patient characteristics 46. In such embodiments, the patient characteristics may be any of the characteristics mentioned previously in the examples above.

In a step 47, tasks are identified from the identified characteristics. The identified tasks are usually tasks that are identified in addition to the tasks associated with clinical pathways (i.e. those identified in the left-hand path in FIG. 4 with steps 42 and 43). Such tasks may be identified from the characteristics using the methods described in any of the examples above.

The paths following the first branch (steps 42 and 43) and the second branch (steps 46 and 47) are merely exemplary, however, and the method 40 may be supplemented by identifying tasks derived from other sources in addition to, or instead of one of the paths shown in FIG. 4.

For example, routine processes such as ward rounding, feeding and washing may be added to the list of tasks that need to be scheduled for medical professionals with respect to a patient. Such tasks may be held in a database of common tasks that are to be performed on certain groups of patients.

In some embodiments, tasks may also be identified from other hospital data, for example, from therapeutic plans (e.g. bespoke plans created by a medical professional that contain tasks for an individual patient). A therapeutic plan may comprise, for example, details of medication that is to be given to the patient, and/or scheduled interventions and procedures.

Once a list of tasks has been generated for all patients that are scheduled to use the medical facility in the defined period (from a combination of tasks associated with clinical pathways and/or associated with other treatment needs, as described above), each task is assigned 44 to a medical professional.

Tasks may be assigned to a medical professional based on, for example, the geographic location of each medical professional, the availability of each medical professional, the skills of each medical professional and/or the tasks usually performed by each medical professional.

In some embodiments, a method may include a step of identifying duplicate tasks in a task list and determining whether a duplicate task needs to be performed more than once. For example, drawing blood and lab tests may be tasks common to more than one pathway. However, there may be overlap in the tests required and therefore one would want to reduce the number of interventions in terms of time, cost and quality of care efficiency by combining the lab tests into a single blood test.

In some embodiments, duplicated tasks are highlighted to a medical professional so that a decision may be taken as to whether the task needs to be performed more than once. In some embodiments one or more duplicated tasks may be combined into a single task.

Once tasks are assigned to a relevant medical professional, a step 45 comprises identifying bottlenecks in the assigned tasks. A bottleneck may be a period of time during which too many tasks are assigned to an individual, or time periods in which the medical facility is likely to be over-stretched. In some embodiments therefore, for each medical professional, the task lists are reviewed and analysed for periods where too much or too little work is assigned.

In some embodiments, identifying bottlenecks comprises taking information from the assignment of tasks and analysing the assigned tasks to determine one or more clashes in a medical professional's schedule, whereby too many tasks have been assigned to the medical professional.

In some embodiments, a medical professional will “accept” (i.e. indicate that they intend to perform) each assigned task. Therefore, the step of identifying bottlenecks may be based on information relating to the number of tasks historically accepted by the medical professional, the number of tasks completed by the medical professional and/or the number of tasks reassigned by the medical professional.

In some embodiments, planning software known in the state of art can be deployed to balance workload or assign additional medical professionals.

In this way, tasks can be identified and schedules can effectively be drawn up based on both standardised clinical pathway data and the real, everyday practice of individual medical facilities, or individual medical practitioners within a medical facility.

Variations to the disclosed embodiments can be understood and effected by those skilled in the art in practicing the principles and systems disclosed herein, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

It should be apparent from the foregoing description that various example embodiments of the invention may be implemented in hardware or firmware. Furthermore, various exemplary embodiments may be implemented as instructions stored on a machine- readable storage medium, which may be read and executed by at least one processor to perform the operations described in detail herein. A machine-readable storage medium may include any mechanism for storing information in a form readable by a machine, such as a personal or laptop computer, a server, or other computing device. Thus, a machine-readable storage medium may include read-only memory (ROM), random-access memory (RAM), magnetic disk storage media, optical storage media, flash-memory devices, and similar storage media.

It should be appreciated by those skilled in the art that any block diagrams herein represent conceptual views of illustrative circuitry embodying the principles of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A computer-implemented method of scheduling a task for a medical professional, comprising:
obtaining patient characteristics associated with a patient;
obtaining test subject characteristics associated with a plurality of test subjects;
identifying a task to be performed in relation to the patient based on the patient characteristics, the test subject characteristics and one or more tasks performed on the test subjects; and
scheduling, using a processor, the identified task for the medical professional.

2. A computer-implemented method as in claim 1 wherein a clinical pathway is assigned to the patient, the clinical pathway having a plurality of branches, each branch including a plurality of tasks; and
wherein the step of identifying comprises:
predicting that a first branch in the clinical pathway will be followed in relation to the patient, based on the patient characteristics, the test subject characteristics and the branches followed for each of the test subjects.

3. A computer-implemented method as in claim 2 wherein predicting that a first branch in a clinical pathway will be followed comprises:
assigning a probability that a first branch will be followed in relation to the patient, based on:
a proportion of the test subjects who followed the first branch of the clinical pathway;
the patient characteristics; and
the test subject characteristics.

4. A computer-implemented method as in claim 3 wherein assigning a probability further comprises:
matching the patient to a subset of the test subjects with similar test subject characteristics to the patient characteristics,
wherein similar comprises:
the test subject characteristics for the subset being identical to the patient characteristics;
the test subject characteristics for the subset being within a predefined threshold of the patient characteristics;
the test subject characteristics for the subset being considered a fuzzy match; or
the test subject characteristics being considered similar to the patient characteristics by a machine learning algorithm.

5. A computer-implemented method as in claim 3 wherein the step of scheduling comprises, scheduling a task from the first branch in the clinical pathway if the probability is higher than a threshold.

6. A computer-implemented method as in claim 2 wherein the step of scheduling comprises notifying the medical professional that the identified task is a predicted task.

7. A computer-implemented method as in claim 1 wherein:
the patient characteristics comprise details of a clinical pathway assigned to the patient;
the test subject characteristics comprise details of one or more clinical pathways assigned to each of the test subjects; and
wherein the step of identifying a task comprises:
identifying a task to be performed in relation to the patient based on tasks performed for a subset of the test subjects who have been assigned to the same clinical pathway as that assigned to the patient.

8. A computer-implemented method as in claim 7 wherein each clinical pathway includes a plurality of tasks to be performed; and
wherein the identified task is a task determined to have been performed in relation to the subset of test subjects, in addition to, or instead of, one or more tasks included in the clinical pathway assigned to the patient.

9. A computer-implemented method as in claim 8 wherein the identified task is not a task included in the clinical pathway assigned to the patient.

10. A computer-implemented method as in claim 8 to further comprising:
obtaining a list of tasks included in the clinical pathway assigned to the patient;
comparing the identified task to the list of tasks; and
determining not to schedule the identified task if the identified task is one of the tasks in the list of tasks.

11. A computer-implemented method as in claim 7 wherein the step of identifying further comprises using a machine learning algorithm to identify the task, based on the patient characteristics, the test subject characteristics and tasks performed on the one or more test subjects.

12. A computer implemented method as in claim 7, further comprising:
receiving additional patient characteristics associated with the patient; and
determining whether the identified task should be performed based on the additional patient characteristics, the test subject characteristics and one or more tasks performed on the test subjects.

13. A computer implemented method as in claim 12, further comprising:
removing the identified task from the schedule of the medical professional, if it is determined that the identified task should not be performed.

14. A computer implemented method as in claim 1, wherein the step of identifying a task comprises comparing the patient characteristics to the test subject characteristics.

15. A computer program product comprising a non-transitory computer readable medium, the computer readable medium having computer readable code embodied therein, the computer readable code being configured such that, on execution by a suitable computer or processor, the computer or processor is caused to perform the method of claim 1.
